(19) United States
(12) Patent Application Publication
(10) Pub. No.: US 2022/0186294 A1
(43) Pub. Date: Jun. 16, 2022

(54) Nanogap Device for Biopolymer Identification

(71) Applicant: Universal Sequencing Technology Corporation, Canton, MA (US)

(72) Inventors: Peiming Zhang, Gilbert, AZ (US); Ming Lei, Sharon, MA (US); Kisup Chung, Slingerlands, NY (US)

(21) Appl. No.: 17/604,046

(22) PCT Filed: Apr. 15, 2020
(86) PCT No.: PCT/US2020/028364

§ 371 Date: Oct. 15, 2021

Related U.S. Application Data
(60) Provisional application No. 62/833,870, filed on Apr. 15, 2019.

(51) Int. Cl. C12Q 1/6809; C12Q 1/6869

(52) U.S. Cl. CPC C12Q 1/6809; C12Q 1/6869

(57) ABSTRACT

This invention provides a device for sequencing and identification of biopolymers electronically.

### FIELD

Embodiments of the present invention are related to nanogap devices for electronic sensing and identification of biopolymers. The biopolymers in the present invention are, but not limited to, DNA, RNA, oligonucleotides, proteins, peptides, polysaccharides, their analogies, either natural or synthetical, etc. In the following disclosure, DNA is used as a material to illustrate the essential framework of the invention.

### BACKGROUND OF THE INVENTION

A nanogap spanning between two electrodes has attracted much attention for use in the development of new DNA sequencing technology. It provides an electronic method to sense biological interactions and biochemical reactions at a single molecule level potentially without molecular labeling, an advantage over the fluorescent detection that requires dye molecules as tags. The nanogap can be fabricated using semiconductor technology, massively produced at a low cost. Besides, its small size warrants a hand-held device that can be used in the point of care.

As a DNA molecule passes through a nanogap between two sharp electrodes consecutively under a voltage bias, each of its nucleotides would modulate the tunneling current across the gap. Thus, by tracing the changes in tunneling currents that feature individual nucleobases, the sequence of the DNA molecule can be readout. Because electron tunneling decays exponentially, a nanogap has to be smaller than 3 nm for electron transport effectively. With the status quo of nanofabrication technology, it is challenging to manufacture such a small nanometer-sized gap on an industrial scale with a high yield and quality.

In this invention, a nanogap can be made larger than 3 nm by bridging it with a conductive nanowire structure, whose conformation is sensitive to its surrounding changes. It functions as a signal transducer with a sensing molecule attached. Thus, this invention provides a functional nanogap device for chemo- and bio-sensing. In particular, this invention provides a nanogap device for DNA sequencing when a DNA polymerase is attached to the nanowire. The sequence of a single DNA molecule can be read out in real-time by recording the electric signals caused by the incorporation of nucleotides to a primer using the target DNA as the template. A nanogap DNA sequencer can be composed of an array of hundred thousand of nanogaps, enabling low cost (<$100) and high throughput real-time (˜1 hour) sequencing of a human genome.

To further improve the conductivity of the nanowire structure, a non-conventional gate electrode is introduced in this invention so that the nanogap can be made even larger to ease the nanogap fabrication and improve signal quality. The introduction of the gate electrode makes the nanogap essentially a FET (field effect transistor) device.

Field-effect transistors have been intensively investigated for their biosensor applications because they can naturally be integrated into portable electronic devices, and also because the field effect is capacitance-related, which is known to be very sensitive to surface changes. Electrostatic interactions in an electrolyte solution are known to extend at most to Debye's screening length λ. It defines the length-scale at which a charged analyte can be electrically probed at the detector interface; Indeed, if a charge resides at a distance further than the A value, it is shielded by the ions of the electrolyte solution. Some reports show how organic FET (OFET) and nanowire FET (NWFET) sensors become “blind” to the target molecule (analyte) when the value of Debye's length is below that of the distance at which the recognition event takes place.^{1, 2} In general, these contributions suggest that the FET detection is only possible at salt concentrations that are low enough so that A is larger than the analyte size.³

In a conventional MOSFET sensor, the gate electrode is covered by an insulating layer. By replacing the insulating material with an electrolyte to covere the gate electrode, the gate electrode becomes sensitive to modulations of the chemical potential in the electrolyte solution.⁴ In the electrolyte-gated FET (EGFET), the FET channel and the gate electrode are in direct contact with the electrolyte. Thus, two electrochemical double layers (EDL) are formed: one at the semiconductor/electrolyte interface, and a second one at the gate electrode/electrolyte interface. As a result, the modulation of the channel potential occurs due to capacitive processes.⁵ This is the main difference between an EGFET and classical MOSFET and OFET, in which the doping of the semiconductor material is responsible for the on/off switching characteristics of the transistor.⁴ One of the main advantages of an EGFET is its comparatively low operating potential (<1 V) which prevents undesired redox reaction or even water splitting, thus enabling applications in an aqueous environment which is evidently important for the detection of important analytes in biological samples. Recently, Nakatsuka et al. have detected small molecules under physiological high-ionic strength conditions using printed ultrathin metal-oxide field-effect transistor arrays modified with DNA aptamers with the electrolyte gating.⁶ Also, the electrolyte gating has been used to measure the single-molecule conductivity.⁷

### BRIEF DESCRIPTION OF THE DRAWINGS

Remarks: All drawings here are just for an illustrative purpose. Their dimensions are not sketched in scale, and the shapes of the elements and connection among them are all illustrative, not representing the real objects.

FIG. 1: Nanogap molecular sensing device using tunable nanostructure without a gate electrode

FIG. 2: Nanogap molecular sensing device with an insulated gate electrode (conventional FET device)

FIG. 3: Nanogap molecular sensing device with a bare gate electrode (electrolyte gated FET (EGFET) device).

FIG. 4: Trapezoidal nanogap (a) with sensing electrode covered on the top; (b) with sensing electrode partially exposed on the top; (c) with an insulated gate electrode and partial top exposed sensing electrode.

FIG. 5: Sensing electrode made of more than one metal, (a) two metals, (b) three metals where metal 2 and metal 3 can be the same or different.

FIG. 6: A schematic diagram of a nanogap device for DNA sequencing by DNA polymerase attached to a conductive DNA origami nanostructure.

FIG. 7: A schematic diagram of a nanogap device for DNA sequencing by DNA polymerase with a universal base molecular tweezer integrated on the DNA nanostructure.

FIG. 8: A schematic diagram of a nanogap device for DNA sequencing by DNA helicase with a nucleobase recognizing molecular tweezer integrated on the DNA nanostructure.

FIG. 9: Chemical structures, calculated DFT structures (B3LYP/6−311+G(2df,2p)), and molecular orbitals of canonical base pairs, base pairs between modified adenine and thymine (in this DFT study, all sugar moieties of the nucleosides are replaced with the methyl group to simplify the calculation).

FIG. 10: effects of substituent groups at adenine on the HOMO energy level of the AT base pair, calculated by DFT in the same way as described in FIG. 9

### SUMMARY OF THE INVENTION

This invention provides a nanogap molecular sensing device for the electronic identification and/or sequencing of biopolymers as well as process recording of biochemical reactions and biological interactions. In one embodiment, a nanogap is about a 10 nm size between two electrodes on a non-conductive substrate (e.g., a silicon substrate) topped by an insulation layer (e.g., silicon nitride or silicon dioxide). The electrodes are fully covered by a (dielectric) insulation layer, or by a chemical passivation monolayer. The electrodes are made of metals, preferably, Platinum (Pt), Palladium (Pd), Gold (Au), Tungsten (W), Copper (Cu), Aluminum (Al), Silver (Ag), Chromium (Cr), Tantalum (Ta), Titanium (Ti) and Titanium nitride (TiN), or conductive carbon materials such as carbon nanotube and graphene, or transition-metal dichalcogenides preferring to MoX₂ (X═S, Se, Te), or doped silicon. For making a functional nanogap device for electronic measurement, a conductive nanostructure of comparable size carrying a sensing molecular moiety is used to bridge the nanogap. In this invention, a tunable conductive DNA nanostructure, such as those disclosed in US Provisionals 62/794,096 and 62/812,736, is suitable for bridging the gap with the same attachment methods disclosed in the two Provisionals. A DNA polymerase, e.g., ϕ29 DNA polymerase, is immobilized onto the DNA nanostructure. For sequencing, a target DNA (template) is subjected to replication by the polymerase in the device. During the replicating process, nucleotides are incorporated into an elongating DNA primer by the DNA polymerase. Mechanistically, the incorporation of a nucleotide into DNA is accompanied by changes in the conformation of the polymerase, which would disturb the conformation of DNA nanostructure. This process results in the fluctuation of electrical currents that can be used as signatures to identify the incorporation of different nucleotides since the conductivity of a DNA molecule is related to its conformation. Alternatively, the DNA nanostructure can be replaced by carbon nanotubes, and those molecular wires simply made of double-stranded DNAs, polypeptides, or other conductive polymers.

In some embodiments of this invention, a nanogap is formed using the conventional FET concept. As illustrated in FIG. 2, a gate electrode layer is constructed underneath the nanogap, and one of the sensing electrodes acts as the source, and another as the drain (they are exchangeable). The addition of a gate electrode reportedly increases the conductivity of the nanogap device^{2,3,11}, allowing higher signal strength than the nanogap without the gate electrode mentioned in the previous embodiment.

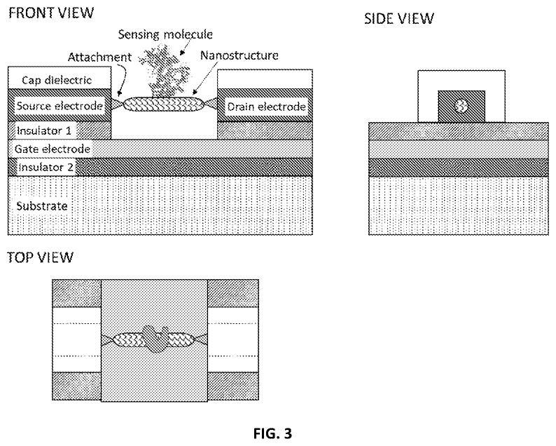

In some embodiments of this invention, as a further improvement for the nanogap device performance, the gate electrode mentioned above, as shown in FIG. 2, is exposed to electrolyte buffer at the nanogap by removing the insulation layer there, as illustrated in FIG. 3. This process creates an EGFET type nanogap device.

For illustrative purpose, the following is an example of how to construct the EGFET nanogap device using nanofabrication technology:

P1. Substrate preparation
Semiconductor or insulating (e.g., glass) substrate

P2. Insulator 2 deposition
SiNx, SiOx, or other dielectric materials prepared by chemical vapor deposition (CVD), atomic layer deposition (ALD), physical vapor deposition (PVD), molecular vapor deposition (MVD), Electroplating, or Spin Coating, etc. A preferred method is a plasma enhanced CVD (PECVD) or low-pressure CVD (LPCVD). The thickness of this layer is usually between 1 nm-10 μm or thicker, preferably 2 nm-100 nm. When the substrate is insulating or non-conductive, this step can be omitted.

P3. Gate electrode deposition
This layer comprises a noble metal such as Au, Pt, Pd, W, Ti, Ta, TiNx, TaNx, Al, Ag, Cr, Cu, and other metals and/or common HK/MG materials used in semiconductor, preferably differing from the sensing electrode for better control of bridging nanostructure attachment. A preferred method is sputtering or evaporation PVD. The thickness of this layer is usually between 2 nm-1 μm or larger, preferably 3 nm-50 nm.

P4. Insulator 1 deposition
SiNx, SiOx, or other dielectric materials are used to prepare this layer, preferably by chemical vapor deposition (CVD), atomic layer deposition (ALD), physical vapor deposition (PVD), molecular vapor deposition (MVD), Electroplating, or Spin Coating, etc. A preferred method is a plasma-enhanced CVD (PECVD) or low-pressure CVD (LPCVD). The dimension of this layer is similar to insulator 2.

P5. Sensing electrode deposition
Common metallic, conductive layers such as Au, Pt, Pd, W, Ti, Ta, Cr, TiNx, TaNx, Al, Ag, and other metals and/or common HK/MG materials using in semiconductor, preferably Pt, Pd, Au, Ti, and TiN. It can be prepared by methods mentioned in P2, but the most preferred methods are sputtering or evaporation PVD. The thickness of this layer is determined by the bridging nanostructure and sensing molecule, usually between 2 nm-1 μm, or thicker, preferably 3 nm-30 nm.

P6. Sensing electrode line patterning
P6.1 EBL (electron beam lithography) with dose 10,000900,000 uC/cm² or EUV (Extreme ultraviolet lithography)
P6.2 Line Etching: PDE (Plasma Dry Etching) or IBE (Ion beam Etching) or ALE (Atomic Layer Etching), stopped on or into the Insulator 1 layer The line width at the nanogap is usually between 5 nm-1 μm, or wider preferably 5 nm-30 nm.

P7. Cap dielectric deposition
SiNx, SiOx, or other dielectric materials are used to prepare the layer, preferably by chemical vapor deposition (CVD), atomic layer deposition (ALD), physical vapor deposition (PVD), molecular vapor deposition (MVD), Electroplating, or Spin Coating, etc. A preferred method is ALD. The thickness of the dielectric layer is usually between 1 nm-1000 nm, preferably 3 nm-20 nm.

P8. Nanogap patterning
P8.1 EBL with dose 10,000900,000 uC/cm² or EUV
P8.2 Gap etching: PDE (Plasma Dry Etching) or IBE (Ion beam Etching) or ALE (Atomic Layer Etching), stopping on or into the gate electrode layer and then cleaning the Insulating layer1 in the gap area

P9. Interconnects & pad patterning
It is processed with Lift-off as well as adding to the normal Litho-Etch process.

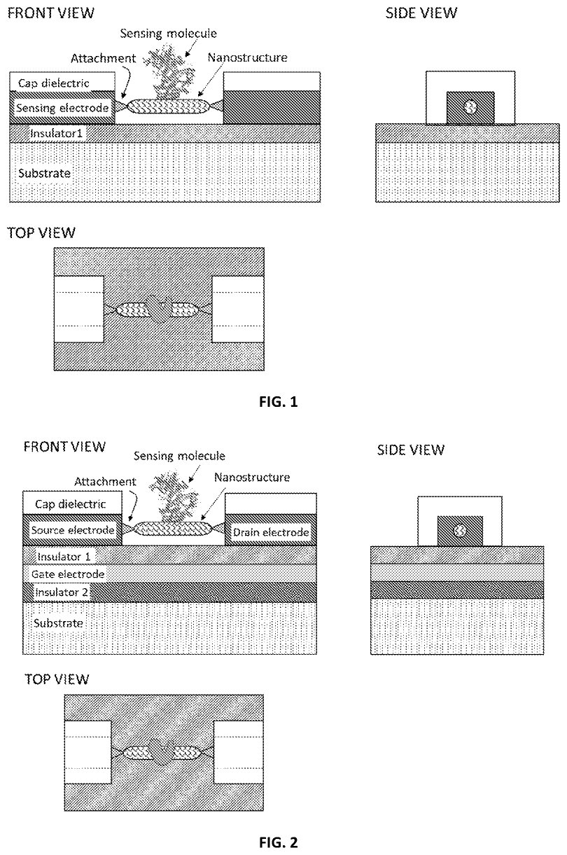

For the construction of the conventional FET nanogap device (FIG. 2), change nanogap etch in Step P8.2 to stop on the Insulator 1 instead of the gate electrode. For the construction of a nanogap device without a gate electrode (FIG. 1), just simply omit Steps P2 and P3.

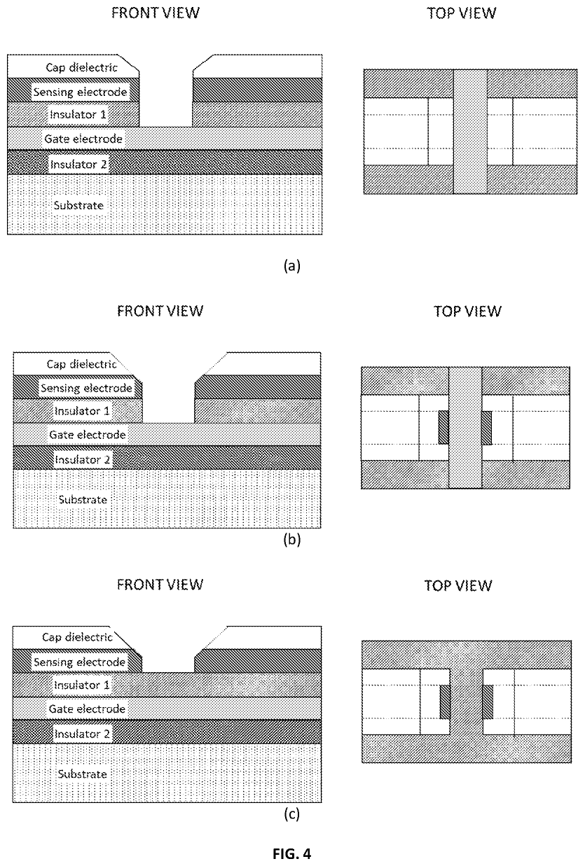

In some embodiments of this invention, the nanogap opening is made wider than the bottom, forming a trapezoidal gap shape, as illustrated in FIGS. 4 (a), (b), and (c). The widened nanogap opening at the top of the gap facilitates the attachment of DNA nanostructure onto the sensing electrodes within the nanogap and the capture and replication of the target DNA by the polymerase. To make the widened opening, at the nanogap fabrication Step 8.2, etch the nanogap at ten or more degrees smaller than the surface normal.

In some embodiments of this invention, the sensing electrode is made of more than one metal layer (see FIG. 5), which provides good adhesion for better electrode fabrication and/or better electrical properties as well as more flexible chemical attachment properties. The two metal layers showing in FIG. 5a may be made of the same thickness or different thickness, each ranging from 1 nm to 1 μm, preferably with the metal layer 1 from 3 nm to 20 nm. The three metal sandwich sensing electrode shown in FIG. 5b may be needed when the center metal needs to be protected or very difficult to adhere to any insulating materials. In the three metal sandwich, metal 2 and metal 3 can be the same material or different materials and can be made very thin (0.5-3 nm) to serve as adhesive layers. In general, the thickness of each layer, as well as the overall electrode thickness, ranges from 3 nm to 30 nm. It may be as thick as several micrometers or even thicker in some cases.

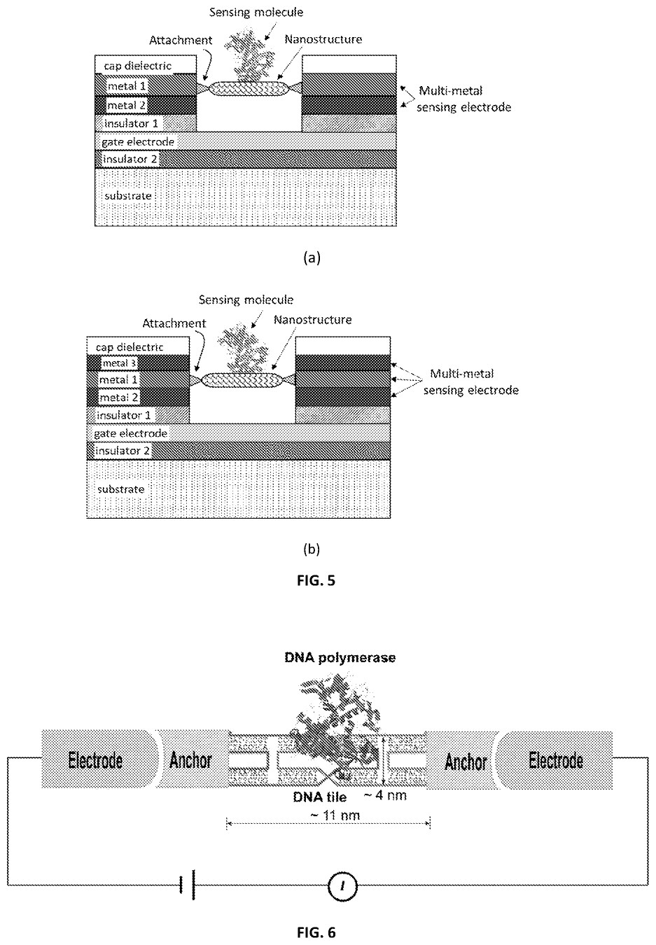

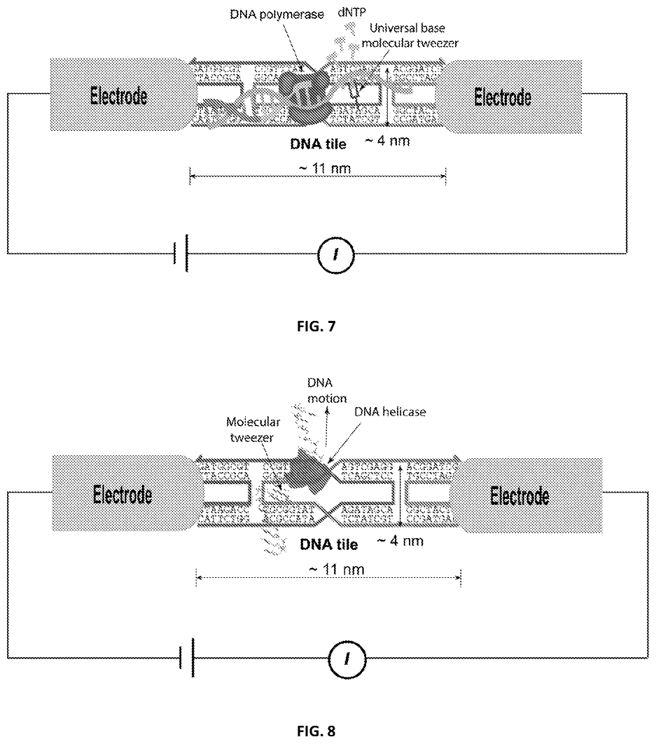

In one embodiment, a nanogap with a size ranging from 5 to 20 nm is fabricated (see FIG. 6). A DNA origami structure is attached to both electrodes to bridge the nanogap, on which a DNA polymerase is immobilized. All relevant methods on the DNA structure and attachment to electrodes are disclosed in U.S. Provisional 62/812,736. The DNA polymerase is selected from the group of Phi29 (ϕ29) DNA polymerase, T7 DNA polymerase, Tag polymerase, DNA polymerase Y, DNA Polymerase Pol I, Pol II, Pol III, Pol IV, and Pol V, Pol α (alpha), Pol β (beta), Pol σ (sigma), Pol λ (lambda), Pol δ (delta), Pol ε (epsilon), Pol μ (mu), Pol I (iota), Pol κ (kappa), pol η (eta), terminal deoxynucleotidyl transferase, telomerase, etc., either natural, mutated or synthesized. DNA is sequenced through polymerase replication in the nanogap device. Alternatively, the DNA nanostructure can be replaced by molecular wires made of double-stranded DNAs, polypeptides, and other conductive polymers, or be replaced by more complex DNA nanostructures.

In some embodiments of the invention, the insulating layers on the gate electrode (Insulator 1) are the material with a high dielectric constant (k >10), including tantalum oxide, strontium titanium oxide, hafnium oxide, hafnium silicon oxide, zirconium oxide, preferring to hafnium oxide. Also, the insulating layer has a thickness of ranging from 2 nm to 1 μm or thicker, preferring to 2 to 100 nm.

In some embodiments of the invention, the nanogap has a dimension of the width ranging from 2 nm to 1 μm, the length ranging from 2 nm to 1 μm, and a depth ranging from 2 nm to 1 μm.

In some embodiments of the invention, a conductive nanowire is attached to both source and drain electrodes to bride the said nanogap. The nanowire has a tunable dimension to accommodate a sensing molecule or multiple sensing molecules with its width to match the sensing molecule's diameter to prevent the sensing molecules from seating on the nanowire's surface in parallel while allowing the individual sensing molecule to be completely placed on the nanowire.

The said nanowire is a nanostructure composed of naturally occurring nucleic acids, synthetic nucleic acids, or their hybrids; naturally occurring peptides, synthetic peptides, or their hybrids; proteins containing unnatural amino acids. These nanostructures contain predefined functions for immobilization of sensing molecules through at one site or multiple sites. These nanostructures also include orthogonal functions for them to be attached to each of the electrodes through one attachment site or multiple sites.

The said sensing molecules are a variety of recognition molecules, including nucleic acid probes, enzymes, receptors, antibodies. All these molecules specifically interact with their targets, which disturb the nanowire's structure resulting in measurable changes in electrical currents.

In some embodiments, the invention provides a nanogap DNA sequencing device. As shown in FIG. 7, the DNA sequencing device is built on a nanogap spanning between two electrodes, bridged by a DNA tile nanostructure functioning as a molecular wire, on which a DNA polymerase is immobilized as a DNA sequence reader. For sequencing, the enzyme incorporates nucleotides to a primer using the target DNA as a template, accompanied by changes in the conformation, which disturbs the underlying DNA nanostructure, resulting in fluctuations in the current flow. To further amplify the changes, a universal base is placed near to the DNA polymerase on the DNA tile, which can equally form base pairs with naturally occurring nucleobases. Thus, when the DNA polymerase moves the DNA molecule, it also disturbs the nucleobase pairing with the universal base, resulting in more changes in the DNA nanostructure and evoking a larger electrical response.

In some other embodiments, the DNA sequencing device comprises a DNA helicase and a nucleobase recognizing molecular tweezer, both immobilized on the DNA nanostructure in the predefined locations (FIG. 8). When a single-stranded DNA passes through the nanogap by the DNA helicase, the nucleobases are captured consecutively by the molecular tweezer. The interactions between the nucleobase and molecular tweezer are different among the naturally occurring nucleobases by the design, so they evoke different electrical responses. Thus, a DNA sequence can be deduced from the electrical signals.

In some embodiments, the DNA nanostructure comprises a different GC/TA ratio. It is well known that the GC base pair is more conductive than the TA base pair.⁸ Thus, the conductivity of the DNA nanostructure can be tuned by changing the GC content. Since the GC base pair is more rigid than the TA, the flexibility of the DNA nanostructure can be increased by increasing the TA content, which results in a DNA nanostructure more responsive to chemical or biological events. For better conductivity of the DNA nanostructure, a GC content of 50% to 95% is necessary, preferably 60% to 80%.

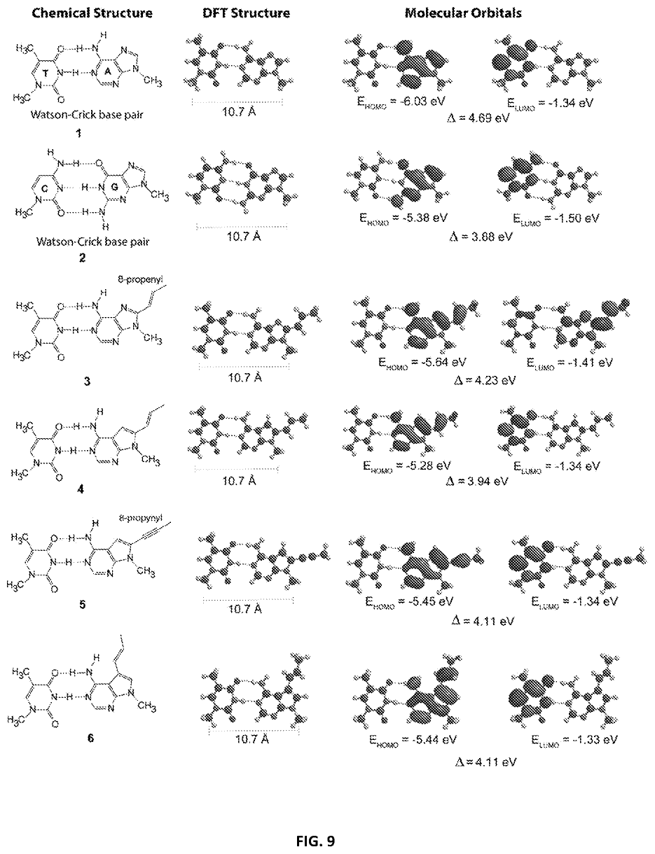

In some embodiments, the DNA nanostructure contains a modified adenine or adenines, which is used to improve the conductivity of DNA nanostructures with their flexibilities maintained (FIG. 9). It has been measured that a GC base pair is ˜3 times more conductive than an AT base pair in a B-form conformation in aqueous solution.⁸ While the conductivity of GC sequences decay linearly with their length, those of TA sequences decay exponentially with their lengths.⁹ These may be explained by the molecular structures of these base pairs. The GC base pair (2, FIG. 9) has a smaller energy gap between its LUMO and HOMO compared to the AT base pair (1, FIG. 9). Thus, the AT base pair becomes a barrier for the electron transfer in DNA. As the electron transports through an electrode-DNA-electrode junction, the process would be the most efficient one around the Fermi level of the metal electrodes (E_{F}). Thus, the molecular orbital (MO) with its energy level that is the closest to the Fermi level of an electrode makes a major contribution to the molecular conductance.¹⁰ Compared to its LUMO, the HOMO of the GC base pair has energy closer to the gold electrode's Fermi level (−5.5 eV), so the DNA molecule conducts through the base G where the HOMO is located (2, FIG. 9). To improve the conductivity of DNA molecules, this invention provides modified adenines with their HOMO energy levels closer to those of the metal electrodes than the naturally occurring adenine. As shown in FIG. 9, the modifications occur at the position 7 and 8 of adenine (see the AT base pair 1 in FIG. 9 for the labeling), which do not affect the modified adenines to form the canonical Watson-Crick base pairs with thymine (T). The invention modifies adenine or 7-dazaadenine using organic groups containing double and triple bonds to form conjugated structures. These molecules can form the base pair with T through hydrogen bonding (3, 4, 5, 6 in FIG. 9) with their HOMOs closer to one of GC base pairs to a different extent.

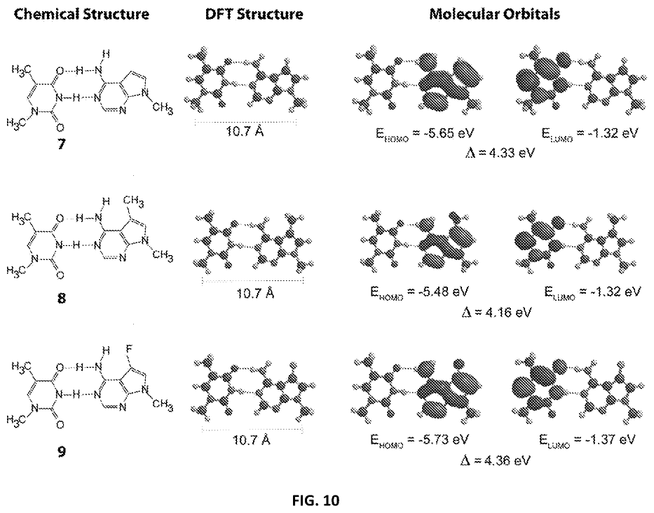

In some embodiments, the invention provides a method to tune the HOMO level of DNA base pairs for tuning the conductivity of DNA. By comparing the AT base pair 1 (FIG. 9) with the base pair 7 (FIG. 10), replacing N at position 7 of adenine by CH elevates the HOMO level energy level of the base pair from −6.03 eV to −5.65 eV. As shown in FIG. 10, replacing the hydrogen of the CH by an electron donor group (EDG) methyl group (CH₃) further increases the HOMO level energy level of the base pair from −5.65 eV to −5.48 eV, whereas replacing the hydrogen of the CH by an electron withdrawing group (EWG) fluorine (F) decreases the HOMO level energy level of the base pair from −5.65 eV to −5.73 eV. Thus, the conductivity of a DNA nanostructure can be tuned by introducing EDG or EWG to those canonical base pairs. Both EDGs and EWGs can be any of substituent groups that can tune the HOMO energy levels and in turn conductivity of DNA.

In some embodiments, the invention provides a device having a universal base concomitantly with DNA polymerase immobilized on the DNA nanostructure. The universal base can indiscriminately base pair with naturally occurring nucleobases. It interacts with single-stranded DNA to slow down its translocation through the DNA polymerase for a uniform synthetic process. The universal bases are those compounds such as triazole-carboxamide for the hydrogen bonding interactions with the naturally occurring nucleobases, and 5-nittroindole for the stacking interactions with the naturally occurring nucleobases.

In other embodiments, the invention provides a device having a molecular tweezer (selected from those disclosed in U.S. Provisional 62/772,837) concomitantly with DNA helicase immobilized on the DNA nanostructure. The helicase translocates DNA to the molecular tweezer for reading out the nucleobases.

In some embodiments, the above-mentioned nanogap DNA sequencing devices and methods are applicable to sequencing RNA and proteins too.

In some embodiments, a nanochip containing an array of nanogaps between 100 to 100 million, preferably between 1,000 to 1 million, is made to satisfy the throughput requirements of biopolymer sensing or sequencing.

In some embodiments, an array of nanogap devices on one chip is divided into multiple regions or modules, and the signals are read out separately from one region to other regions by separate signal recording units to overcome the bandwidth and sampling frequency limits of a single recording unit.

### General Remarks

All publications, patents, and other documents mentioned herein are incorporated by reference in their entirety.

Unless defined otherwise, all technical and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will readily appear to those skilled in the art. The invention in its broader aspects is therefore not limited to the specific details, representative device, apparatus and method, and illustrative example shown and described. Accordingly, departures may be made from such details without departing from the spirit of applicant's general inventive concept.

### REFERENCES

1. Ishikawa, F. N.; Curreli, M.; Chang, H.-K.; Chen, P.-C.; Zhang, R.; Cote, R. J.; Thompson, M. E.; Zhou, a. C., A Calibration Method for Nanowire Biosensors to Suppress Device-to-Device Variation. ACS nano 2009, 12, 3969-3976.
2. Hammock, M. L.; Knopfmacher, O.; Naab, B. D.; Tok, J. B. H.; Bao, Z., Investigation of Protein Detection Parameters Using Nanofunctionalized Organic Field-Effect Transistors. ACS Nano 2013, 7(5), 3970-3980.
3. Stern, E.; Wagner, R.; Sigworth, F. J.; Breaker, R.; Fahmy, T. M.; Reed, M. A., Importance of the Debye Screening Length on Nanowire Field Effect Transistor Sensors. Nano Lett. 2007, 7 (11), 3405-3409.
4. Kergoat, L.; Piro, B.; Berggren, M.; Horowitz, G.; Pham, M.-C., Advances in organic transistor-based biosensors: from organic electrochemical transistors to electrolyte-gated organic field-effect transistors. Anal Bioanal Chem 2012, 402, 1813-1826.
5. Palazzo, G.; Tullio, D. D.; Magliulo, M.; Mallardi, A.; Intranuovo, F.; Mulla, M. Y.; Favia, P.; Vikholm-Lundin, I.; Torsi, L., Detection Beyond Debye's Length with an Electrolyte-Gated Organic Field-Effect Transistor. Adv. Mater. 2015, 27, 911-916.
6. Nakatsuka, N.; Yang, K.-A.; Abendroth, J. M.; Cheung, K. M.; Xu, X.; Yang, H.; Zhao, C.; Zhu, B.; Rim, Y. S.; Yang, Y.; Weiss, P. S.; Stojanović, M. N.; Andrews, A. M., Aptamer-field-effect transistors overcome Debye length limitations for small-molecule sensing. Science 2018, 362, 319-324.
7. Li, Y.; Buerkle, M.; Li, G.; Rostamian, A.; Wang, H.; Wang, Z.; Bowler, D. R.; Miyazaki, T.; Xiang, L.; Asai, Y.; Zhou, G.; Tao, N., Gate controlling of quantum interference and direct observation of anti-resonances in single molecule charge transport. Nature Materials 2019, 18, 357-363.
8. Hihath, J., Xu, B., Zhang, P. & Tao, N. Study of single-nucleotide polymorphisms by means of electrical conductance measurements. Proc Natl Acad Sci USA 102, 16979-16983, doi:10.1073/pnas.0505175102 (2005).
9. Xu, B., Zhang, P., Li, X. & Tao, N. Direct Conductance Measurement of Single DNA Molecules in Aqueous Solution. Nano Lett. 4, 1105-1108 (2004).
10. Furuhata, T. et al. Highly Conductive Nucleotide Analogue Facilitates Base-Calling in QuantumTunneling-Based DNA Sequencing. ACS nano (2019).
11. Liu, S.; Zhang, X.; Luo, W.; Wang, Z.; Guo, X.; Steigerwald, M. L., & Fang, X. Single-molecule detection of proteins using aptamer-functionalized molecular electronic devices. Communications, Angew. Chem. Int. Ed. 2011, 50, 2496-2502, doi:10.1002/anie.201006469.

What is claimed is:

1. A system for identification, characterization, and/or sequencing of a biopolymer comprising,
a. a substrate;
b. a nanogap formed by a first electrode and a second electrode placed next to each other on the substrate;
c. a nanostructure configured to have a dimension about or comparable to the size of the nanogap and configured to bridge the nanogap by attaching one end to the first electrode and another end to the second electrode through a chemical bond;
d. a sensing molecule attached to the nanostructure configured to interact with the biopolymer and perform a biochemical reaction;
e. a gate electrode placed between the nanogap and the substrate; and
f. a first insulation layer separating the gate electrode and the nanogap together with the first and the second electrodes.

2. The system of claim 1, further comprising
a. a second insulation layer separating the gate electrode and the substrate, wherein the second insulation layer is optional when the substrate is non-conductive or coated with a non-conductive material; and
b. a cap dielectric layer covering the first and the second electrodes.

3. The system of claim 1, further comprising
a. a bias voltage that is applied between the first electrode and the second electrode;
b. a reference voltage that is applied to the gate electrode;
c. a device configured to record a current fluctuation through the nanostructure resulting from a distortion within the nanostructure caused by a conformation change initiated by the sensing molecule; and
d. a software for data analysis configured to identify, characterize and/or sequence the biopolymer or a subunit of the biopolymer.

4. The system of claim 1, wherein the biopolymer is selected from the group consisting of DNA, RNA, oligonucleotide, protein, peptide, polysaccharide, either natural, modified or synthesized of any of the aforementioned biopolymers, and a combination thereof.

5. The system of claim 1, wherein the sensing molecule is selected from the group consisting of a nucleic acid probe, an enzyme, a receptor, and an antibody, either native, mutated, expressed, or synthesized, and a combination thereof.

6. The system of claim 5, wherein the enzyme is selected from the group consisting of a DNA polymerase, a RNA polymerase, a DNA helicase, a DNA ligase, a DNA exonuclease, a reverse transcriptase, a terminal deoxynucleotidyl transferase, a telomerase, a RNA primase, a ribosome, a sucrase, a lactase, either native, mutated, expressed, or synthesized of any of the aforementioned enzymes, and a combination thereof.

7. The system of claim 6, wherein the DNA polymerase is selected from the group consisting of ϕ29 DNA polymerase, T7 DNA polymerase, Taq polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ε (epsilon), Pol μ (mu), Pol I (iota), Pol κ (kappa), Pol η (eta), either native, mutated, expressed, or synthesized of any of the aforementioned enzymes, and a combination thereof.

8. The system of claim 1, wherein the nanostructure comprises a conductive DNA structure or a conductive RNA structure comprising natural, modified or synthetic nucleic acids, and comprising a double-stranded DNA, a DNA/RNA duplex, a DNA origami structure, a DNA nanostructure of any shape, or a combination thereof.

9. The system of claim 8, wherein the DNA or RNA structure comprises a universal base configured to base-pair with a natural nucleobase substantially indiscriminately, wherein the universal base interacts with the natural nucleobase through either hydrogen bonding or base stacking.

10. The system of claim 8, wherein the DNA or RNA structure comprises a GC content of about 50% to about 95%.

11. The system of claim 8, wherein the DNA or RNA structure comprises a GC content of about 60% to about 80%.

12. The system of claim 8, wherein the DNA or RNA structure comprises a modified adenine that improves the conductivity of the nanostructure without substantially affecting the AT base pairing.

13. The system of claim 8, wherein the conductivity of DNA or RNA structure is configured to be tunable by modifying the adenine of the AT base pair by (1) replacing N at position 7 with CH; (2) further replacing the hydrogen of the CH with an electron donor group comprising a methyl group, CH₃ or with an electron-withdrawing group comprising a fluorine, F; (3) further replacing the hydrogen of the CH with an alkene group or an alkyne group either at position 7 or 8.

14. The system of claim 1, wherein the nanostructure is a conductive polypeptide or a polypeptide structure, made of either natural, modified or synthetic amino acids, or any conductive polymer, or a combination thereof.

15. The system of claim 1, further comprising a molecular tweezer configured to be attached to the nanostructure next to the sensing molecule, and configured to assist in the identification, characterization and/or sequencing of the biopolymer.

16. The system of claim 15, wherein the sensing molecule comprises a DNA helicase.

17. The system of claim 2, further comprising a chemical passivation layer on top of the cap dielectric layer.

18. The system of claim 1, further comprising a chemical passivation layer on top of the first and the second electrodes.

19. The system of claim 1, wherein the first insulation layer comprises one of the following configuration:
a. a substantially continuous coverage across the nanogap, covering the gap electrode underneath it, and
b. a substantially discontinuous coverage across the nanogap, exposing the gap electrode underneath it at the nanogap site.

20. The system of claim 1, wherein the first insulation layer comprises a dielectric material with a dielectric constant higher than about 10 comprising strontium titanium oxide, hafnium oxide, hafnium silicon oxide, zirconium oxide, or a combination thereof.

21. The system of claim 1, wherein the first and the second electrodes comprise a noble metal comprising Platinum (Pt), Palladium (Pd), Gold (Au), Tungsten (W), Copper (Cu), Aluminum (Al), Silver (Ag), Chromium (Cr), Tantalum (Ta), Titanium (Ti), Titanium nitrides (TiNx), or Tantalum nitrides (TaNx), or a conductive carbon material such as a carbon nanotube or a graphene, or a transition-metal dichalcogenide in the form of MoX₂ (X═S, Se, Te), or a doped silicon, or a combination thereof.

22. The system of claim 1, where the gate electrode is made of a common metallic material, including but not limited to Gold (Au), Platinum (Pt), Palladium (Pd), Tungsten (W), Titanium (Ti), Tantalum (Ta), Titanium nitrides (TiNx), Tantalum nitrides (TaNx), Aluminum (Al), Silver (Ag), Chromium (Cr), Copper (Cu), or a common semiconductor HK/MG materials, and a combination thereof.

23. The system of claims 1 and 2, wherein:
a. the nanogap comprises a width ranging from about 2 nm to about 1000 nm, a length from about 2 nm to about 1000 nm, and a depth from about 2 nm to about 1000 nm;
b. the first and the second electrodes comprise a thickness substantially equal to the depth of the nanogap and a width substantially equal to the width of the nanogap;
c. the gap electrode comprises a thickness of about 2 nm to about 1000 nm;
d. the cap dielectric layer comprises a thickness of about 1 nm to about 1000 nm; and/or
e. the first insulation layer and the second insulation layer each comprises a thickness from about 1 nm to about 1000 nm.

24. The system of claims 1 and 2, wherein:
a. the nanogap comprises a width ranging from about 5 nm to about 30 nm, a length from about 5 nm to about 20 nm, and a depth from about 3 nm to about 30 nm;
b. the first and the second electrodes comprise a thickness substantially equal to the depth of the nanogap and a width substantially equal to the width of the nanogap at the nanogap;
c. the gap electrode comprises a thickness of about 3 nm to about 50 nm;
d. the cap dielectric layer comprises a thickness of about 3 nm to about 20 nm; and/or
e. the first insulation layer and the second insulation layer each comprises a thickness of about 2 nm to about 100 nm.

25. The system of claim 1, wherein the first and the second electrodes comprise two or more metal layers of the same or different materials with a combined thickness substantially equal to the depth of the nanogap.

26. The system of claim 1, wherein the first and the second electrodes comprise of three metal sandwich layers with a mid-layer comprising a different material from a top layer and a bottom layer and a thickness of the top and bottom layers ranging from about 0.5 nm to about 3 nm, and a total thickness substantially equal to the depth of the nanogap.

27. The system of claim 1, wherein a wall of the nanogap is tapered with an opening of the nanogap being wider than the bottom.

28. The system of claim 27, wherein the tapering of the wall of the nanogap comprises about 10 degrees or more relative to a normal of the substrate surface.

29. The system of claims 1, 2, 3, 15, 17 and 18 comprises a plurality of nanogaps, each comprising all components and any feature associated with a single nanogap.

30. The system of claim 29, wherein the plurality of nanogaps comprises an array of about 100 to about 100 million nanogaps, preferably between about 10,000 to nearly 1 million nanogaps.

31. The system of claims 15 to 30, wherein the nanostructure comprises a carbon nanotube.

32. A method for identification, characterization, and/or sequencing of a biopolymer comprising,
a. providing a substrate;
b. building a second insulation layer on the substrate, wherein the second insulation layer is optional when the substrate is non-conductive or coated with a non-conductive material;
c. building a gate electrode layer on the second insulation layer, or directly on the substrate when the second insulation layer is absent;
d. building a first insulation layer on top of the gate electrode layer;
e. building a first electrode and a second electrode on the first insulation layer, and placing them substantially next to each other to form a nanogap;
f. providing a nanostructure comprising a dimension substantially comparable to the nanogap and is configured to bridge the nanogap by attaching one end to the first electrode and another end to the second electrode through a chemical bond; and
g. providing a sensing molecule configured to interact with the biopolymer and perform a biochemical reaction, and attaching the sensing molecule to the nanostructure at a predefined location.

33. The method of claim 32, further comprising
a. applying a bias voltage between the first electrode and the second electrode;
b. applying a reference voltage to the gate electrode;
c. providing a device configured to record a current fluctuation through the nanostructure resulting from a distortion within the nanostructure caused by a conformation change initiated by the sensing molecule attached to the nanostructure; and
d. providing a software for data analysis configured to identify, characterize, and/or sequence the biopolymer or a subunit of the biopolymer.

34. The method of claim 32, wherein the biopolymer is selected from the group consisting of DNA, RNA, oligonucleotide, protein, peptide, polysaccharide, either natural, modified or synthesized of any of the aforementioned biopolymers, and a combination thereof.

35. The method of claim 32, wherein the sensing molecule is selected from the group consisting of nucleic acid probes, enzymes, receptors, and antibodies, either native, mutated, expressed, or synthesized, and a combination thereof.

36. The method of claim 35, wherein the enzyme is selected from the group consisting of a DNA polymerase, a RNA polymerase, a DNA helicase, a DNA ligase, a DNA exonuclease, a reverse transcriptase, a RNA primase, a terminal deoxynucleotidyl transferase, a telomerase, a ribosome, a sucrase, a lactase, either native, mutated, expressed, or synthesized of any of the aforementioned enzymes, and a combination thereof.

37. The method of claim 36, wherein the DNA polymerase is selected from the group consisting of ϕ29 DNA polymerase, T7 DNA polymerase, Taq polymerase, DNA polymerase Y, DNA Polymerase Pol I, Pol II, Pol III, Pol IV and Pol V, Pol α (alpha), Pol β (beta), Pol σ (sigma), Pol λ (lambda), Pol δ (delta), Pol ε (epsilon), Pol μ (mu), Pol I (iota), Pol κ (kappa), Pol η (eta), either native, mutated, expressed, or synthesized of any of the aforementioned enzymes, and a combination thereof.

38. The method of claim 32, wherein the nanostructure is a conductive DNA or a RNA structure made of either natural, modified or synthetic nucleic acids, and comprising a double-stranded DNA, a DNA/RNA duplex, a DNA origami structure, a DNA nanostructure of any shape, and a combination thereof.

39. The method of claim 38, wherein the DNA or RNA structure comprises a universal base configured to base-pair with a natural nucleobase substantially indiscriminately, wherein the universal base interacts with the natural nucleobase through either a hydrogen bonding or a base stacking.

40. The method of claim 38, wherein the DNA or RNA structure comprises a GC content of about 50% to about 95%.

41. The method of claim 38, wherein the DNA or RNA structure comprises a GC content of 60% to 80%.

42. The method of claim 38, wherein the DNA or RNA structure comprises a modified adenine configured to improve the conductivity of the nanostructure without substantially affecting the AT base pairing.

43. The method of claim 38, wherein the conductivity of the DNA or RNA structure is configured to be tunable by modifying the adenine of the AT base pair by (1) replacing N at position 7 with CH; (2) further replacing the hydrogen of the CH with an electron donor group comprising a methyl group, CH₃ or with an electron-withdrawing group comprising fluorine, F; (3) further replacing the hydrogen of the CH with an alkene group or an alkyne group either at position 7 or 8.

44. The method of claim 32, wherein the nanostructure comprises a conductive polypeptide or a polypeptide structure, made of either natural, modified or synthetic amino acids, or any conductive polymer, or a combination thereof.

45. The method of claim 32, further comprising providing a molecular tweezer, and attaching it to the nanostructure at a predefined location and being configured to assist in the identification, characterization and/or sequencing of the biopolymer.

46. The method of claim 45, wherein the sensing molecule is a DNA helicase.

47. The method of claim 32, further comprising covering the first and the second electrodes with a cap dielectric layer with the ends of the electrodes being exposed at the nanogap, or alternatively covering the first and the second electrodes with a chemical passivation layer.

48. The method of claim 47, further comprising covering the cap dielectric layer with a chemical passivation layer.

49. The method of claim 32, wherein the first insulation layer comprises one of the following configurations:
a. a substantially continuous coverage across the nanogap, covering the gap electrode underneath it, and
b. a substantially discontinuous coverage across the nanogap, exposing the gap electrode underneath it at the nanogap site.

50. The method of claim 32, wherein the first insulation layer comprises a dielectric material with a dielectric constant higher than about 10 comprising tantalum oxide, strontium titanium oxide, hafnium oxide, hafnium silicon oxide, zirconium oxide, and a combination thereof.

51. The method of claim 32, wherein the first and the second electrodes comprise a noble metal, comprising Platinum (Pt), Palladium (Pd), Gold (Au), Tungsten (W), Copper (Cu), Aluminum (Al), Silver (Ag), Chromium (Cr), Tantalum (Ta), Titanium (Ti), Titanium nitrides (TiNx), Tantalum nitrides (TaNx), or a conductive carbon material such as a carbon nanotube and a graphene, or a transition-metal dichalcogenide in the form of MoX₂ (X═S, Se, Te), or a doped silicon, or a combination thereof.

52. The method of claim 32, where the gate electrode comprises a common metallic material, comprising Gold (Au), Platinum (Pt), Palladium (Pd), Tungsten (W), Titanium (Ti), Tantalum (Ta), Titanium nitrides (TiNx), Tantalum nitrides (TaNx), Aluminum (Al), Silver (Ag), Chromium (Cr), Copper (Cu), or a common semiconductor HK/MG materials, and a combination thereof.

53. The method of claims 32 and 47, wherein at the site of the nanogap:
a. the nanogap comprises a width ranging from about 2 nm to about 1000 nm, a length from about 2 nm to about 1000 nm, and a depth from about 2 nm to about 1000 nm;
b. the first and the second electrodes comprise a thickness substantially equal to the depth of the nanogap and a width substantially equal to the width of the nanogap;
c. the gap electrode comprise a thickness of about 2 nm to about 1000 nm at the nanogap site;
d. the cap dielectric layer comprise a thickness of about 1 nm to about 1000 nm; and
e. the first insulation layer and the second insulation layer each comprises a thickness from about 1 nm to about 1000 nm.

54. The method of claims 32 and 47, wherein at the site of the nanogap:
a. the nanogap comprises a width ranging from about 5 nm to about 30 nm, a length from about 5 nm to about 20 nm, and a depth from about 3 nm to about 30 nm;
b. the first and the second electrodes has a thickness substantially equal to the depth of the nanogap and a width substantially equal to the width of the nanogap;
c. the gap electrode comprises a thickness of about 3 nm to about 50 nm;
d. the cap dielectric layer comprises a thickness of about 3 nm to about 20 nm; and
e. the first insulation layer and the second insulation layer each comprises a thickness of about 2 nm to about 100 nm.

55. The method of claim 32, wherein the first and the second electrodes comprise two or more metal layers of the same or different materials with a combined thickness substantially equal to the depth of the nanogap.

56. The method of claim 32, wherein the first and the second electrodes are made of three metal sandwich layers with a mid-layer comprising different material from a top layer and a bottom layer and the thickness of the top and bottom layers ranging from about 0.5 nm to about 3 nm, and a total thickness substantially equal to the depth of the nanogap.

57. The method of claim 32, wherein a wall of the nanogap is substantially tapered with an opening of the nanogap being wider than the bottom.

58. The method of claim 57, wherein the tapering of the wall of the nanogap is about 10 degrees or more relative to a normal of the substrate surface.

59. The method of claim 32, wherein each of the insulation layers and the electrode layers is fabricated separately using a semiconductor material deposition method, comprising chemical vapor deposition (CVD), atomic layer deposition (ALD), physical vapor deposition (PVD), molecular vapor deposition (MVD), Electroplating, or Spin Coating, or a combination thereof.

60. The method of claim 32, wherein the insulation layers are fabricated using either a plasma-enhanced CVD (PECVD) or a low pressure CVD (LPCVD) method.

61. The method of claim 32, wherein the electrodes are fabricated using a sputtering method.

62. The method of claim 32, wherein the first and the second electrodes and the nanogap are fabricated using EBL (electron beam lithography) or EUV (Extreme ultraviolet lithography), or PDE (plasma dry etching) or IBE (ion beam etching) or ALE (atomic layer etching).
